(19) United States
(12) Patent Application Publication
(10) Pub. No.: US 2010/0247637 A1
(43) Pub. Date: Sep. 30, 2010

(54) COMPOSITIONS AND PHARMACEUTICAL FORMS FOR THE ORAL ADMINISTRATION OF THYROID HORMONES ABLE TO COMBAT THE ACTION OF SEQUESTRANTS IN THE GASTROINTESTINAL TRACT

(76) Inventors: Giorgio Zoppetti, Milano (IT); Paoio Mazzucchelli, Venegona Inferiore (IT); Marta Riva, Luino (IT)

Correspondence Address: STETINA BRUNDA GARRED & BRUCKER, 75 ENTERPRISE, SUITE 250, ALISO VIEJO, CA, 92656, US

(21) Appl. No.: 12/428,550

(22) Filed: Apr. 23, 2009

(30) Foreign Application Priority Data
Mar. 24, 2009 (IT) MI2009A000466

(51) Int. Cl. A61K 9/48; A61K 47/26; A61K 47/42; A61K 47/40; A61K 31/197

(52) U.S. Cl. 424/456; 514/778; 514/774; 514/777; 514/567

(57) ABSTRACT

The invention concerns the use of a substance chosen among animal or vegetable gelatine, peptones, dextrins, native or modified cyclodextrins, starch, and starch hydrolysates in the preparation of pharmaceutical forms for the oral administration of thyroid hormones to combat the action of sequestrant agents present in the gastrointestinal tract, and related pharmaceutical forms.

### CROSS-REFERENCE TO RELATED APPLICATIONS

Not Applicable

### STATEMENT RE: FEDERALLY SPONSORED RESEARCH/DEVELOPMENT

Not Applicable

### FIELD OF THE INVENTION

This invention concerns thyroid hormone-based pharmaceutical formulations for oral administration.

### BACKGROUND

The hormones produced by the thyroid cells are released into the blood circulation and act on the body metabolism, thereby increasing oxygen consumption and the production of heat with a consequent increase in body temperature, stimulating protein synthesis and positivising the nitrogen balance, increasing gluconeogenesis and glycogenolysis, stimulating synthesis, mobilisation and catabolism of cholesterol and lipids in general. The thyroid hormones increase the speed of cell oxidative processes and regulate the metabolism of most tissues. In general, at low doses we obtain a mainly anabolic effect, whilst at high doses, we see a catabolic action. This biphasic action is evident with regards to the metabolism of the glycogen, proteins and lipids.

In the event of physiological deficiency, treatment based on the administration of thyroid hormones is necessary. T3 (lyothyronine, or O-(4-hydroxy-3-iodophenyl)-3.5-diiodo-L-tyrosine) and T4 (levothyroxine, or O-(4-hydroxy-3.5-diiodophenyl)-3.5-diiodo-L-tyrosine) are commonly used thyroid hormones, employed as such or in the form of hydrated or sodium salts for various different therapeutic applications and obtained by synthesis or by extraction from animal glands.

Therapeutic treatment of deficiency of these hormones yields satisfactory results by taking T3 and T4 (or their respective sodium salts). T3 and T4 in particular are mainly used in the treatment of hypothyroidism.

Very often, therapy involving the administration of thyroid hormones continues throughout the patient's life. Dosage must be established individually. Generally speaking, a low dose is initially prescribed. The quantity is then gradually increased until the clinical evaluation and laboratory tests show that optimal response has been obtained from the treated organism. The dose required to obtain this response is then maintained. The patient's general health and age, and the severity and duration of hypothyroidism symptoms determine the initial dose and the speed with which the dose is then taken to the definitive level. It is extremely important to increase doses very gradually in patients suffering from myxoedema or cardiovascular diseases, in order to prevent an onset of angina, myocardial infarction or ictus.

This is the reason why T3 and T4, their respective sodium salts, and their combination (Liotrix) are administered orally, and specifically in tablet form. This means that by controlling the frequency with which they are taken, or the choice of different doses, administration is tailored to suit the patient's individual situation.

An exact dosage is extremely important as underdosing may result in insufficient response, and therefore in hypothyroidism, whilst overdosing may result in toxic manifestations of hyperthyroidism such as, for example, cardiac pain, palpitations or cardiac arrhythmias. In patients suffering from coronary disease, even the slightest increase in the dose of levothyroxine can be very dangerous.

As such, due to the risks associated with both overdosing and underdosing of thyroid hormones in general, it is absolutely critical that patients can rely on forms for administration that are consistent in terms of title and bioavailability.

Lyothyronine (T3) and levothyroxine (T4) are currently marketed in solid oral form, and mainly as soft gelatine capsules and tablets, or in oral drops.

However, it has been found that the pharmacokinetics of T4 administered orally in tablet form are affected by food substances present in the stomach, with a sequestrant effect. Specifically, Benvenga et al (Thyroid Vol. 18, 3-2008, 293-301) have conducted both in vitro and in vivo tests showing that the assumption of dietary fibres or coffee can reduce the plasmatic kinetic curve of the T4.

The simultaneous administration of other pharmaceutical products used for other pathologies, such as antacids like aluminium hydroxide, magnesium hydroxide or sucralfate, can also reduce the plasmatic kinetics of the T4 administered in oral tablets, as compared with the standard conditions considering no food or pharmaceutical products.

The study specifically showed a subtraction effect of the active ingredient where the stomach contained dietary fibres such as bran. Bran fibres mainly consist of cellulose that is not digested by the enzyme systems in the human digestive tract, thereby encouraging intestinal motility. As the cellulose is eliminated directly with faeces, the fraction of T4 sequestered by the cellulose is thus subtracted from the permeation of the intestinal membrane, depressing bioavailability and therapeutic action.

### BRIEF SUMMARY

According to this invention, it has been surprisingly found that such negative sequestrant effect can be efficiently combated by formulating the thyroid hormones in forms for oral administration, with the addition of a substance chosen from: animal or vegetable native or hydrolysed gelatine, peptones, dextrins, native or modified cyclodextrins, starch, and starch hydrolysates with high oligomer content.

The invention therefore concerns the use of a substance chosen among animal or vegetable gelatine, peptones, dextrins, native or modified cyclodextrins, starch, and starch hydrolysates in the preparation of pharmaceutical forms for the oral administration of thyroid hormones to combat the action of sequestrant agents present in the gastrointestinal tract, and the pharmaceutical compositions thus prepared.

### DETAILED DESCRIPTION

Such sequestrant agents present in the gastrointestinal tract are mainly understood as deriving from diet, such as, for example, fibres like bran, or coffee, or from pharmaceutical products used for other therapies employed at the same time, such as antacids.

The thus-prepared formulations of thyroid hormones for oral use include both solid forms, namely capsules, and liquid forms, such as solutions to be administered in drops or in disposable containers.

The characteristics and advantages of this invention are explained in detail in the following description.

The following examples are provided to aid disclosure, and do not limit this invention.

### EXAMPLE 1

Preparation of soft gelatine capsules containing T4

Soft gelatine capsule with a shell and fill, or inner phase, containing 100 μg levothyroxine T4 have been prepared as follows:

a) preparation of the shell mixture:

Components and quantities for preparation and relative percentage:

| | | |
|---|---|---|
| Gelatine 150 bloom | 46.8 kg | 39.0% |
| Anhydrous glycerine | 27.6 kg | 23.0% |
| Purified water | 45.6 kg | 38.0% |

27.6 kg of anhydrous glycerine are added to 45.6 kg of purified water in a 150 litre turbo emulsifier (Olsa-Italia). The mixture is well shaken and taken to a temperature of 70° C. 46.8 kg gelatine is then added and the mixture continually shaken for a further 15-60 minutes. The mass is then de-aerated by applying a progressive vacuum until reaching a value ranging between +0.8 and −0.9 bars.

b) preparation of the fill (for 100 mcg dose with 50 mg fill)

Components and quantities for preparation and relative percentage:

| | | |
|---|---|---|
| Hydrolysed gelatine | 3.5 kg | 35.0% |
| Gelatine 80 bloom | 0.5 kg | 5.0% |
| Glycerin 85% | 3.5 kg | 35.0% |
| Purified water | 2.48 kg | 24.8% |
| Sodium levothyroxine (T4) | 0.02 kg | 0.2% |

The levothyroxine is dissolved in 2.5 kg of 85% glycerine, mixing with a mechanical stomacher until fully dissolved.

Separately, in a 25 litre turbo emulsifier (Olsa Italia), 2.48 kg of water and 1.0 kg of 85% glycerine are suctioned. This mix is heated to 65° C.±5°. Having reached the temperature, 3.5 kg of hydrolysed gelatine and 0.5 kg of gelatine 80 bloom are suctioned, mixed and de-aerated until a clear product is obtained with no agglomerates.

At this point, the product, which has been kept at the temperature of 65° C.±5°, is cooled to 45° C.±3° C. Once this temperature has been reached, the solution containing the levothyroxine is added, mixing for at least 30 minutes.

During this phase, both the vacuum and cooling are kept active, in order to take the product to the temperature of 38° C.±2°.

The product obtained is discharged into a thermostat container and kept at 38° C.±2°.

Capsule Preparation

Soft gelatine capsules were prepared with a 10 oval format and in accordance with the method known as the ‘Rotary Die Process’.

Capsules with the following characteristics were obtained:

average capsule weight: 205 mg±10%

residual humidity: 4.0-10%

content in T4: 0.100 mg/capsule equal to 100.0% d.d.

### EXAMPLE 2

Preparation of a glycerol-ethanol solution of sodium levothyroxine (T4) with the addition of gelatine.

Components and quantities for a 25 litre preparation:

| | |
|---|---|
| Sodium levothyroxine (T4) | 2.625 g |
| Glycerol (85%) | 21.525 kg |
| Ethanol (96%) | 6.100 kg |
| Hydrolysed gelatine | 2.500 kg |

90% of the ethanol (5.49 litres) are poured into a 10-litre steel container equipped with blade stomacher and lid. Whilst being shaken, the T4 is added. Shake slowly, keeping a flow of nitrogen until fully dissolved. The glycerol and hydrolysed gelatine, together with the ethanol solution containing the T4, are poured into a 25-litre turbo emulsifier (Olsa-Italia). Wash the 10-litre container with the remaining ethanol (0.61 litres) and pour into the 25-litre turbo emulsifier. Continue to shake reasonably gently for 15 minutes in nitrogen atmosphere, and protected from the light.

### EXAMPLE 3

Preparation of a glycerol-ethanol solution of sodium levothyroxine (T4) with the addition of starch hydrolysates

Components and quantities for a 25 litre preparation:

| | | |
|---|---|---|
| Sodium levothyroxine (T4) | 2.625 | g |
| Glycerol (85%) | 21.525 | kg |
| Ethanol (96%) | 6.100 | kg |
| Starch hydrolysate (Glucidex-Roquette-France) | 2.500 | kg |

The final formula is obtained as described for example 2, using the starch hydrolysate in lieu of the gelatine.

Experimental study on the sequestrant effect of fibres; dissolution test With the aim of testing the effect of the formulae prepared in accordance with the previous examples, a dissolution test was carried out with increasing quantities of microcrystalline cellulose fibre (1 and 2% p/V).

The test is based on the fact that an increasing concentration of cellulose in the system involves a proportional decrease of the T4 in the solution after filtration. The effect is entirely inhibited by the addition of a surfactant like sodium dodecyl sulphate SDS 0.2%. We can therefore conclude that the missing T4 is sequestered by the cellulose.

The dissolution test for the solid oral forms is used as a quality test that shows the dissolution of the oral form and the quantitative liberation of the active ingredient available for the pharmacokinetics (dissolution test EuPh 6.0 (2.9.3) and USP 30 (711). In the case of solid oral T4 tablets, the existing dissolution methods (USP 30-Levothyroxine sodium tablets) set out, as per protocol, the use of hydrochloric acid 0.01 N as a dissolution buffer in the presence of SDS. Given, however, the effect inhibiting sequester by the surfactant, as discussed above, the method must be altered, eliminating the SDS from the buffer. The following equipment is used for the test:

Sotax Dissolver

Equipment: Basket

Rpm: 100

Medium: HCl 0.01 N

Medium volume: 500 ml

Medium temperature: 37° C.

Sample times: 10-15-20-25-30-35-45-80

Sample volume: 1 ml (without restoral)

Chromatographic column: Symmetry Shield RP 18 5 μm-150 mm×3.9 mm

Column temperature: 35° C.

Mobile phase: H2O/CH3CN/H3PO4

Flow: 1 ml/min

Wavelength: 225 nm

Run time: 30 minutes

Injection volume: 400 μl.

Figure Graphs

The figures of the attached drawings compare the dissolution curves with the percentage quantity of T4 passed in the solution on the y-axis, and the time (t, minutes) on the x-axis, as follows:

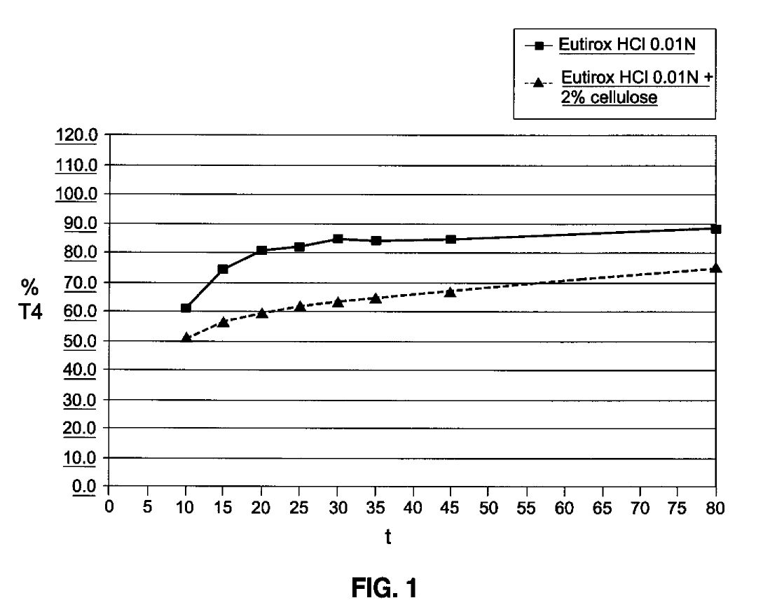

FIG. 1 compares the two dissolution curves of a famous T4 tablet available on the market (Eutirox®), respectively without and with cellulose 2%. The curves are obtained with the USP method modified without the addition of SDS.

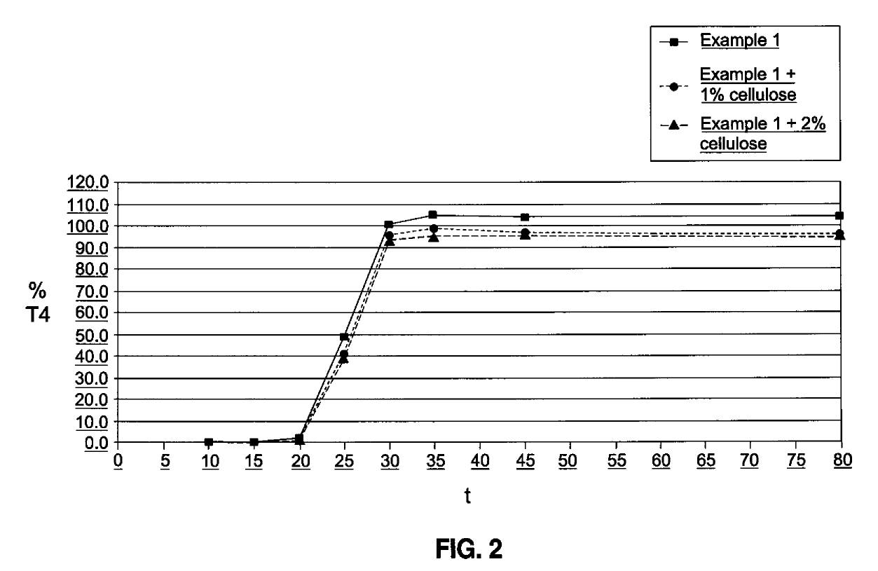

FIG. 2 compares the three dissolution curves of a T4 formula according to example 1 of this invention, respectively without and with cellulose, in one case 1% and in the second case 2%.

In FIG. 1, the sequestrant effect of the cellulose with regards to T4 is clearly visible, with a dissolution profile lowered by approximately 10 up to 20% as compared with the dissolution profile without cellulose.

In FIG. 2, the graph shows that in the presence of 1 or 2% microcrystalline cellulose, the T4 dissolution profile does not vary significantly as compared with the absence of fibre, as the curves can be almost superimposed.

As such, it is clear how the invention allows for the attainment of the aim discussed initially.

What is claimed is:

1. Use of a substance chosen among animal or vegetable gelatine, peptones, dextrins, native or modified cyclodextrins, starch, and starch hydrolysates in the preparation of pharmaceutical forms for the oral administration of thyroid hormones to combat the action of sequestrant agents present in the gastrointestinal tract.

2. Use according to claim 1 where the said substance is gelatine, for the preparation of soft gelatine capsules.

3. Use according to claim 1 for the preparation of a liquid form in solution to be administered in drops.

4. Use according to claim 1 for the preparation of a liquid form in single-dose solution.

5. Pharmaceutical form for the oral administration of thyroid hormones to combat the action of sequestrant agents present in the gastrointestinal tract, characterised by containing a substance chosen from animal or vegetable gelatine, peptones, dextrins, native or modified cyclodextrins, starch, and starch hydrolysates.

6. Pharmaceutical form according to claim 5 characterised by being a soft gelatine capsule.

7. Pharmaceutical form according to claim 6 characterised by the fact that said soft gelatine capsule includes a shell obtained from a mix of gelatine, glycerine and water, and an inner phase, or fill, obtained from a mix of gelatine, glycerine, water and sodium levothyroxine (T4).

8. Pharmaceutical form according to claim 5 characterised by the fact of being a solution to be administered in drops.

9. Pharmaceutical form according to claim 5 characterised by the fact of being a single-dose solution.

10. Pharmaceutical form according to claim 5 characterised by the fact of being a glycerol-ethanol solution of sodium levothyroxine (T4) with the addition of gelatine.

11. Pharmaceutical form according to claim 5 characterised by the fact of being a glycerol-ethanol solution of sodium levothyroxine (T4) with the addition of starch hydrolysate.

12. Method of combatting the action of sequestrant agents present in the gastrointestinal tract of patients in need of thyroid hormones which would depress their bioavailability and therapeutic action, wherein the said thyroid hormones are prepared in pharmaceutical forms for oral administration to which a substance chosen among animal or vegetable gelatine, peptones, dextrins, native or modified cyclodextrins, starch, and starch hydrolysates is added.

13. Method according to claim 12 where the said substance is gelatine and a soft gelatine capsule is prepared.

14. Method according to claim 12 wherein a liquid form in solution to be administered in drops is prepared.

15. Method according to claim 12 wherein a liquid form in single-dose solution is prepared.
